(19) United States
(12) Patent Application Publication
(10) Pub. No.: US 2002/0031842 A1
(43) Pub. Date: Mar. 14, 2002

(54) Immunoassay testing kit and containers

(76) Inventors: Ricky T. Smethers, Fremont, CA (US); William B. Greger, San Francisco, CA (US); Wendy R. Whitcomb, San Francisco, CA (US)

Correspondence Address: Quarles & Brady LLP, 411 East Wisconsin Avenue, Milwaukee, WI, 53202, US

(21) Appl. No.: 09/877,873

(22) Filed: Aug. 16, 2001

Related U.S. Application Data
(63) Continuation of application No. 09/109,803, filed on Jul. 2, 1998.

(51) Int. Cl. C12M 1/34; G01N 33/543

(52) U.S. Cl. 436/518; 435/287.2

(57) ABSTRACT

A kit for use in an immunoassay testing device that detects a binding reaction between a pair of first and second substances, particularly biogenic substances, which specifically bind together allowing deposition of electrically conductive particles which fully or partially completing an essentially open electrical circuit. More particularly, the kit provides self contained reagent and testing packages that are less contaminant prone and subject to minimal waste. The kit has a self contained reagent package and a test cartridge that are accessible by the testing device. The reagent package has cavities for reagents and diluting fluids. The test cartridge has a cavity for the test specimen and circuitry that is closed upon deposition of electrically conductive particles following the binding reaction.

### CROSS REFERENCES TO RELATED APPLICATIONS

This application is a continuation of U.S. patent application Ser. No. 09/109,803 filed on Jul. 2, 1998.

### STATEMENT REGARDING FEDERALLY SPONSORED RESEARCH

Not Applicable

### FIELD OF THE INVENTION

The field of invention is electrical immunoassay testing, more particularly containers for simultaneous immunoassay testing.

### BACKGROUND OF THE INVENTION

Electrical immunoassay testing methods have been developed to determine the presence of foreign substances, microorganisms, or endogenous substances. As a result, these testing methods may be used to detect, bacteria, viruses, or substances relating to drug abuse, human hormones, nucleic acids and the like. Such methods detect a change in electrical properties, such as current, voltage, or resistance. The change in electrical properties indicate that an antigen-antibody reaction has occurred.

One particular electrical immunoassay method described in U.S. Pat. No. 5,284,748 provides a method for detecting a binding reaction between a pair of first and second substances, particularly biogenic substances, which specifically bind together causing full or partial completion of an essentially open electrical circuit. This method is especially useful for detecting antigens in the fluids or tissues of humans or animals. Antigens are chemicals that when introduced to the body provoke production of an antibody. Antigens include drugs, toxins, hormones, allergens, tumor markers, factors, enzymes, steroids, nucleotides and other substances. U.S. Pat. No. 5,284,748 is fully incorporated herein by reference.

The mechanics of implementing the above method, however, have been limited to a manual single batch process that is not commercially viable. A technician must perform the test on an individual sample using multiple reagents and other fluids from individual containers, carefully transferring the reagents to the sample container to avoid contamination and waste.

Automated immunoassay testing devices incorporating the testing method disclosed in U.S. Pat. No. 5,284,748 have not been disclosed heretofore. The method requires multiple transfers of reagents and other fluids to a test specimen in a test cartridge. In addition, the container holding the test sample requires special circuitry that changes resistivity in the presence of the binding reaction disclosed in U.S. Pat. No. 5,284,748.

### BRIEF SUMMARY OF THE INVENTION

The present invention provides a kit for use in an immunoassay testing device that detects a binding reaction between a pair of first and second substances, particularly biogenic substances, which specifically bind together causing full or partial completion of an essentially open electrical circuit. More particularly, the kit provides self contained reagent and testing packages that are less contaminant prone and subject to minimal waste. The kit has a self contained reagent package and a test cartridge that are accessible by the testing device. The reagent package has cavities for reagents and diluting fluids. The test cartridge has a cavity for the test specimen and circuitry that is closed in the presence of the binding reaction when an electrical current is applied to contacts at the top of the cartridge. Thus accomplishing a general objective of providing a self-contained test kit that minimizes waste.

Another object of the present invention is to provide a prepackaged reagent package that can be used in the immunoassay testing device without the risk of contamination. This is accomplished by providing a container having multiple cavities formed in the top containing reagents, rinsing fluid, and for receiving aspirated fluid. The top is sealed by a penetratable foil film.

Still another object of the present invention is to provide a test cartridge that can be used in an automated immunoassay testing device. This is accomplished by mounting a resistor on a container cavity sidewall that is compatible with the immunoassay testing method.

The foregoing and other objects and advantages of the invention will appear from the following description. In the description, reference is made to the accompanying drawings which form a part hereof, and in which there is shown by way of illustration a preferred embodiment of the invention.

### BRIEF DESCRIPTION OF THE DRAWINGS

FIG. 1 is a perspective view of an automated immunoassay testing device which employs the present invention;

FIG. 2 is a perspective view of the automated immunoassay testing device of FIG. 1 with the housing removed;

FIG. 3 is a top plan view of embodiments of the present invention on a carousel in the automated immunoassay testing device of FIG. 1;

FIG. 4 is a top perspective view of a first embodiment of the invention used in the automated immunoassay testing device of FIG. 1;

FIG. 5 is a bottom perspective view of the embodiment of FIG. 4;

FIG. 6 is a top plan view of the embodiment of FIG. 4;

FIG. 7 is a top perspective view of a second embodiment of the invention used in the automated immunoassay testing device of FIG. 1;

FIG. 8 is a top plan view of the embodiment of FIG. 7;

FIG. 9 is a side plan view of the embodiment of FIG. 7; and

FIG. 10 is a perspective view of a resistor sheet used in the embodiment of FIG. 7.

### DETAILED DESCRIPTION OF THE PREFERRED EMBODIMENTS

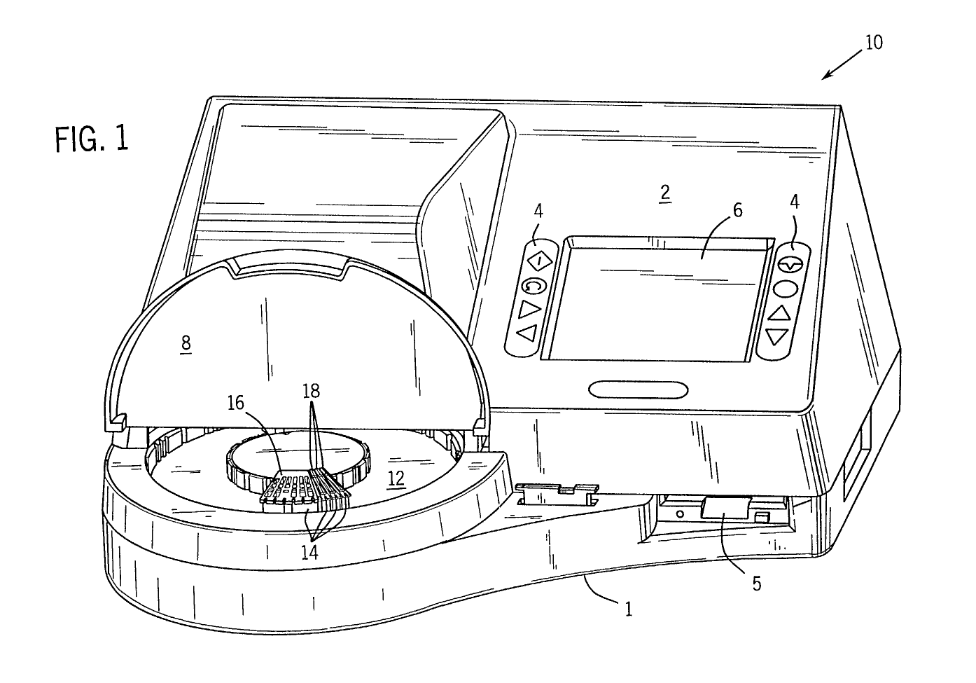

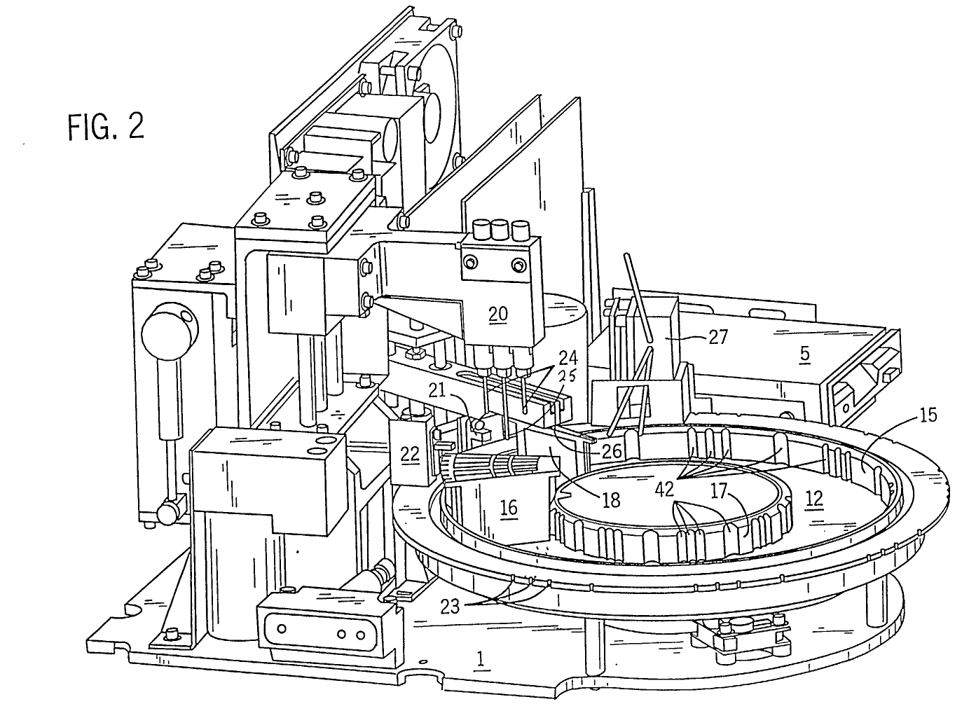

Referring particularly to FIGS. 1 and 2, an immunoassay testing device 10 that detects a binding reaction between a pair of first and second substances, particularly biogenic substances, which specifically bind together causing full or partial completion of an essentially open electrical circuit employs embodiments of the present invention. The testing device 10 is mounted on a base 1 and includes an aluminum housing 2 that supports a keyboard 4, a floppy disk drive 5, and a display 6 on its front surface. A pivotally mounted cover 8 provides access to a circular carousel 12, disposed within the housing 2. The carousel 12 is a conveying mechanism that indexes the containers 14 to various positions within the testing device 10.

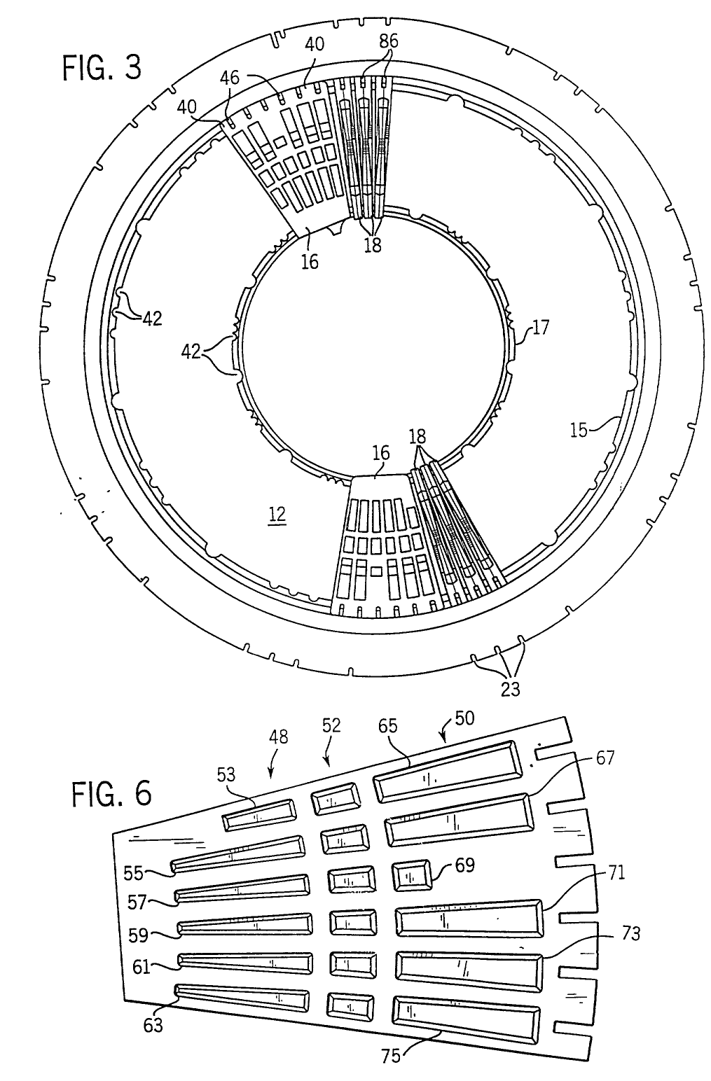

As shown in FIG. 3, containers 14 , such as reagent packages 16 containing fluid reagents used in the testing process and test cartridges 18 for use with testing specimens, forming a kit are placed on the carousel 12. The carousel 12 has indents 42 on an inner wall 15 and outer wall 17 that receive corresponding tabs on the containers 14. The indents 42 define predetermined container locations on the carousel 12. The containers 14 are disposed between the carousel walls 15 and 17. Preferably, up to 9 reagents packages 16 and 27 test cartridges 18 can be placed on the carousel for simultaneous automated testing. Preferably, each reagent package 16 can provide reagents for up to three adjacent test cartridges 18.

Following placement of each container 14 on the carousel 12, the rotating carousel 12 conveys one of the containers 16 to a position disposed beneath a fluidics mechanism 20 to initiate the immunoassay testing procedure. The carousel index position is determined by a first optical sensing unit 21 that detects indents 23 formed on the carousel periphery. The containers 14 are indexed by the carousel 12 in cooperation with a second optical sensing unit 22 adjacent to the carousel that senses a locating mechanism on a container 14.

As shown in FIG. 2, the fluidics mechanism 20 is arranged to cooperate with the reagent package 16 and test cartridge 18 as the containers 14 are indexed on the carousel 12. The fluidics mechanism 20 is mounted for vertical movement and has one center syringe-type needle 25 and two outer syringe-type needles 24 disposed above the carousel 12 between the carousel inner and outer walls. When a container 14 is indexed such that it is disposed beneath the fluidics mechanism 20, the mechanism 20 lowers the needles 24, 25 to access cavities in the cartridge 18 or package 16. As is later described herein, the immunoassay testing procedure requires the fluidics mechanism needles 24 to continually interact with a reagent package 16 and at least one test cartridge 18.

Reagents are drawn from the reagent package 16 using an aspiration pump (not shown). The pump draws fluids into one of the two outer needles 24 and dispenses it into the test cartridge 18 through the same needle 24. The center needle 25 is an aspiration needle that provides an air path allowing the outer needles 24 to draw in reagents. Reagents that have been dispensed into the test cartridge 18 are removed from the cartridge 18 by one of the outer needles 24 and aspirated into an external containment bottle (not shown).

Spring loaded electrical probes 26 are disposed above the carousel 12 and mounted for engagement of electrical contacts at the top of the test cartridge. Although electrical contacts at the top of the test cartridge are described, the contacts may be anywhere on the cartridge, such as the cartridge side, that is accessible by probes adapted for the contact location. The probes 26 apply an electrical current to the electrical contacts for measuring electrical resistivity of circuitry on a resistor sheet 103 mounted in the test cartridge 18.

In one form of the assay, before testing begins, the probes 26 engage the contacts on the test cartridge 18 to determine a base resistivity. Upon completion of the immunoassay test, the test cartridges 18 are again indexed beneath the probes 26 for engagement with the contacts to determine whether a change in resistivity has occurred.

A microprocessor based control system (not shown) controls the movement of all mechanisms in the testing device 10 and accepts inputs, such as expiration dates, container identifiers, and test procedures, for processing and display from an internal bar code reader 27, the floppy disk drive 5, and an optional external bar code reader. The control system verifies that the correct reagent packages 16 will be used for the assigned cartridges 18 and that the containers 14 are not used past their expiration date. Additionally, the test procedure identified for the test cartridge specimen contained within the cartridge 18 is provided to the control system and displayed to the user on the display 6.

The internal bar code scanner 27 reads expiration dates and container identifiers that are encoded on a bar code mounted on the containers 14 and provides this information to the control system for verification and, if appropriate, display. The internal bar code scanner 27 mounted adjacent to the carousel 12 reads the external bar code mounted on the rear wall of each container 14. The bar code is mounted by adhesives or the like and identifies the container 14 to the control system.

The optional external bar code reader (not shown) is connected to the control system by a standard interface known in the art, such as an RS-232 connection. The external bar code reader reads a second bar code affixed to a container sidewall and identifies the container and specimen for user tracking.

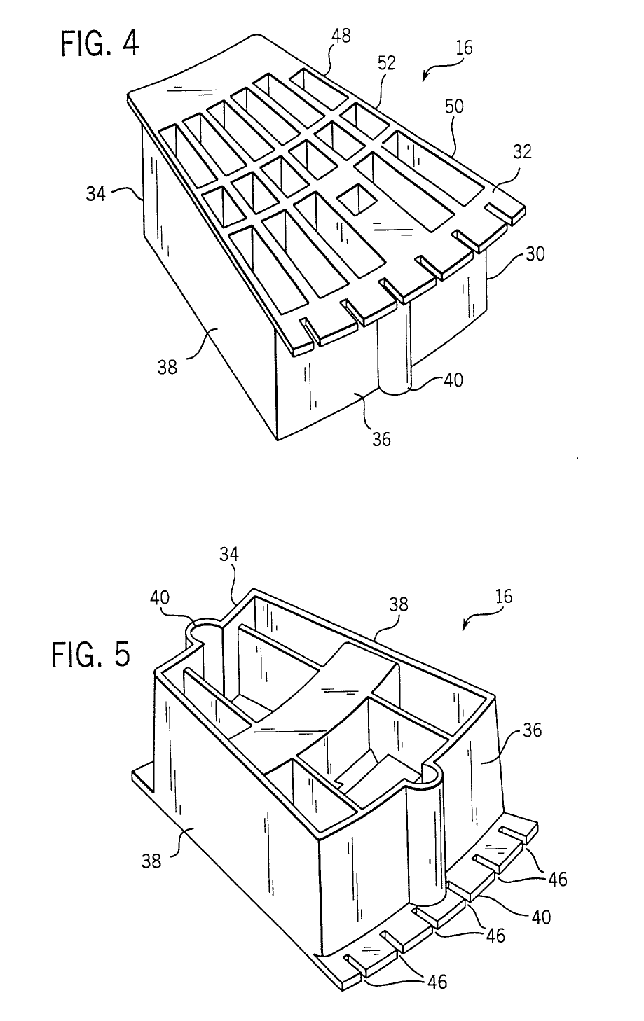

A generally trapezoidal reagent package 16 shown in FIGS. 4-6 has a base 30 and a top 32. A penetratable film (not shown), such as foil, protects the reagents from contamination and spilling during shipment. Preferably, the reagent package 16 is molded from a plastic material that does not react with reagents contained therein, such as polypropylene, polyethylene or the like. Light sensitive reagents are protected by a non-light transmissive color of the container, such as black.

The base 30, as shown in FIG. 5, has a front wall 34, rear wall 36, and two sidewalls 38. Preferably, the base 30 is a segment of an annulus. The annulus is defined by the carousel inner and outer walls 15 and 17. The base front and rear walls 34 and 36 are shaped to conform with the carousel walls 15 and 17. The base sidewalls 38 provide exterior flat surfaces for affixing labels or bar codes by adhesive or other means.

Tabs 40 formed in the front and rear walls 34 and 36 are received by corresponding indents 42 formed in the carousel inner and outer walls 15 and 17. The tabs 40 and corresponding indents 42 ensure proper positioning of the package 16 on the carousel 12 as it is indexed beneath the fluidics mechanism 20 and to other positions in the testing device 10. Although, tabs 40 on the package front and rear wall 34 and 36 are described, other positioning mechanism may be used, such as indents in the cartridge and tabs on the carousel. Additionally, alternatives such as, one or more tabs, indents, or combination thereof may be used on only the front wall or rear wall, rather than on both walls.

The reagent package top 32, shown in FIGS. 4 and 6, is flat with a plurality of cavities 53-75 formed therein. The top 32 extends past the front and rear walls 34 and 36 of the base 30 providing finger holds for lifting and inserting the package 16 in and out of the carousel 12. The top 32 extending past the rear wall 35 has six indents 46 formed therein strategically located for optically sensing the container position relative to the cavities 44. Although, indents sensed by an optical sensor are described, other locating mechanisms known in the art may be used.

Looking particularly at FIG. 6, the reagent package has eighteen upwardly facing cavities 53-75 arranged in three rows for various uses. The first row 48 and third row 50 have six cavities each with volumetric capacities for containing volumes of liquid reagents ranging from 385 to 1600 microliters. The differing volumetric capacities eliminates wasting unused reagents when the cavity volume is sized for the specific reagents used. The second row 52 of cavities disposed between the first and third row 48 and 50 contain distilled water for cleaning the needles 24 to eliminate any cross contamination of fluid during the testing procedure. Each cavity in the center row 52 is a distillation well having a volumetric capacity of 900 microliters. Each time an outer needle 24 draws a reagent from the reagent package 16, the center needle 25 is dipped into the distilled water in a dilution well.

The specificity of the liquids contained within the cavities is determined by the specific test that will be conducted. In one embodiment in which the reagent container 16 is used in testing for the presence of a pathogen, such as Escherichia coli, cavity 53 has a volume of 500 microliters and is empty, cavity 55 contains approximately 700 microliters of a buffer, such as tris(hydroxymethyl)arninomethane (generally known in the art as Tris), cavity 57 contains approximately 700 microliters of hydroquinone, cavity 59 contains approximately 700 microliters of distilled water, cavity 61 has a volume of 700 microliters and is empty, cavity 63 contains approximately 700 microliters of gold-conjugated antibody, cavity 65 contains approximately 1600 microliters of Tris, cavity 67 contains approximately 1600 microliters of distilled H₂O, cavity 69 contains approximately 385 microliters of silver lactate, cavity 71 contains approximately 1600 microliters of distilled H₂O, cavity 73 contains approximately 1600 microliters of distilled H₂O, and cavity 75 contains approximately 1600 microliters of Tris. The above volumes are approximate and used for illustration purposes. Different reagents and different volumes of the reagent may be substituted without departing from the scope of the present invention.

Preferably, specific reagents are provided in the reagent package sealed by a sheet of foil for use by the technician. A prepackaged reagent package eliminates the potential for contamination. Waste is reduced by providing the fluids in specific quantities sufficient for a specific test of a specific number of test specimens.

Preferably, one or more test cartridges 18 are inserted into the carousel 12 adjacent to the reagent package 16 along an axis of motion, as shown in FIGS. 1-3. Preferably, the first test cartridge is directly adjacent to the reagent package sidewall. Additional test cartridges are directly adjacent to the test cartridges being serviced by the reagent package. It is preferred, but not required, to place the reagent package and test cartridges serviced by the reagent package directly adjacent to each other along the axis of motion. Placement of the test cartridges adjacent to the reagent package reduces the potential for contamination or spillage of transferred fluids.

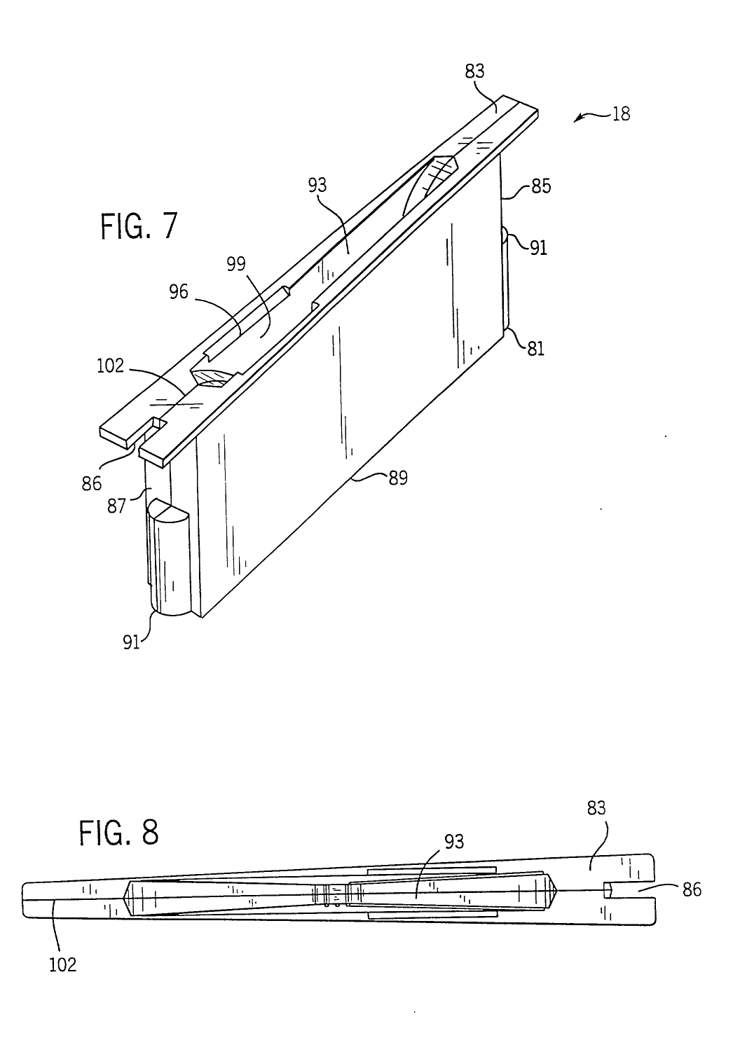

As shown in FIGS. 7 and 8, a generally trapezoidal test cartridge has a base 81 and a top 83. A front wall 85 and rear wall 87 of the base 81 are shaped to conform with the carousel tray inner and outer wall 15 and 17. Base sidewalls 89 provide exterior flat surfaces for affixing labels or bar codes by adhesive or other means.

Round tabs 91 formed in the front and rear walls 83 and 85 are received by corresponding indents 42 in the inner wall 15 and outer wall 17 of the carousel 12. The tabs 89 and corresponding indents 42 ensure proper positioning of the cartridge 18 as it is indexed by the carousel 12. Alternative positioning mechanisms may be used to position the container as described for the first embodiment.

The test cartridge top 83, shown in FIG. 7, is flat with a single upwardly facing cavity 93 therein. The cartridge top 83 extends past the base front and rear walls 85 and 87 providing finger holds for lifting and inserting the cartridge 18 in and out of the carousel. A single indent 86 in the top portion extending past the rear wall 87 allows the optical sensing unit 22 to determine the cartridge 18 position on the carousel 12. Again, alternatives locating mechanisms may be used position the container as described for the reagent package.

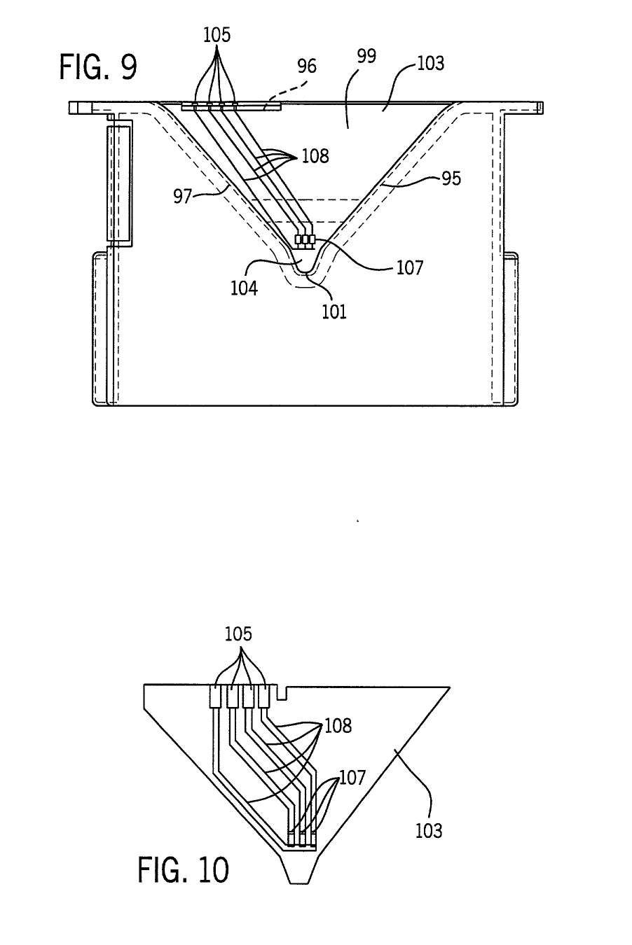

Looking particularly at FIGS. 8 and 9, the test cartridge cavity 93 has a front wall 95, rear wall 97, a pair of sidewalls 99, and a bottom wall 101. The cavity top is substantially defined by the base walls 85, 87 and 89. The cavity front and rear walls 95 and 97 slope inwardly and downwardly towards a receptacle 104 formed in the cavity bottom wall 101. In one embodiment, chamfers 96 formed along the top of the cavity sidewalls 99 expose electrical contacts 105 on a resistor sheet 103 to the spring loaded electrical probes 26. Although a test cartridge having a resistor sheet mounted therein is described, other methods of joining the resistors to a test cartridge, such as molding the resistors having exposed contacts into the side of the cartridge, may be employed to practice the invention.

Preferably, the test cartridge 18 is molded plastic, such as an acrylic or the like, formed from two symmetrical halves joined together at a central plane 102. Although the cartridge 18 could also be a single molded piece, joining two halves simplifies mounting resistor sheets 103 to interior cavity sidewalls 99 as described herein. The halves are joined by conventional means such as ultrasonic welding, bonding, or elastomer seal. Preferably, an elastomer seal, such as santoprene or epoxy, is used. However, even a more rigid seal such as cyanoacrylate will work.

A disposable resistor sheet 103, shown in FIGS. 9 and 10, having circuitry for engagement by the testing device 10 is adhesively mounted to the interior of each cavity sidewall 99. The resistor sheet 103 is, preferably, disposable mylar with conductors 108, such as silver tracings or the like, that electrically connect contacts 105 at the top of the resistor sheet 103 to three parallel resistors 107, such as carbon black or the like, at the bottom. Each resistor 107 on a single sheet is substantially identical having a resistance of at least 150 ohms.

A layer of antigen or binding agent, such as an antibody, deposited over each resistor 107 and a portion of the conductors 108 on each end of the resistor 107 provides a binding surface for a molecule or organism of interest in the test sample. Subsequent sequential application of reagents from the reagent pack 16 leads to the deposition of electrically conductive particles bypassing the resistor 107 creating a closed circuit. The reaction chemistry and various antigen-antibody combinations are disclosed in U.S. Pat. No. 5,284,748, which has been incorporated herein by reference.

It should be understood, however, that only one resistor sheet 103 is necessary to perform the test. By mounting a resistor sheet 103 on each cavity interior sidewall 99, a test for two different types of bacteria, such as Escherichia coli and Salmonella, may be performed on the same sample simultaneously. Upon completion of the test, the resistivity of the three resistors is averaged to maintain test reliability. Three resistors on a single resistor sheet are preferred, however, only a single resistor is necessary to practice the invention.

In use, the external bar code affixed to the sidewall of the cartridge is read by the external bar code reader. The cartridge then is placed on the carousel 12 of the testing device 10. The test cartridge 18 is then indexed by the carousel 12 past the internal bar code reader and then beneath the spring loaded electrical contacts 26. The contacts 26 engage the electrical contacts 105 on the resistor sheet 103 in the cartridge 18 to obtain a base reading prior to testing.

Once the base reading is obtained, the cartridge is indexed to a position accessible by the user and the user adds a specimen to the cartridge by pipetting or other methods of adding a specific volume known in the art. The carousel 12 then indexes the reagent package beneath the fluidics mechanism 20. The mechanism 20 lowers the needles 24, 25 piercing the foil film covering the reagent package 16. The containers 14 are alternately indexed beneath the fluidics mechanism 20 allowing the needles 24 to transfer reagents between the reagent package 16 and test cartridge 18. Diluting fluid, such as distilled water, contained in one or more of the reagent package cavities 44 is used to cleanse the aspiration needle 25 between transfers to avoid contamination.

### EXAMPLE I

In the following example, the automated immunoassay device 10 performs the test for Escherichia coli present in a test specimen. Preferably, the carousel 12 contains at least one reagent package 16 having three adjacent test cartridges 18 and the testing device 10 follows the following procedure:

1) Read resistivity of each resistor sheet;

2) Add bacterial sample to each test cartridge,

3) Incubate test cartridges for 60 minutes;

4) Pick up 113 μl of Tris from cavity 65;

5) Aspirate needles into the containment bottle;

6) Pick up 113 μl of Tris from cavity 65 and dispense into test cartridge; repeat for each test cartridge;

7) Aspirate Tris from each test cartridge into the containment bottle;

8) Pick up 113 μl of Tris from cavity 65 and dispense into test cartridge; repeat for each test cartridge;

9) Aspirate Tris from each test cartridge into the containment bottle;

10) Pick up 140 μl Gold-conjugated antibody from cavity 63 and dispense into test cartridge; repeat for each test cartridge;

11) Incubate test cartridges for 30 minutes;

12) Pick up 110 μl of Tris from cavity 55;

13) Aspirate Tris from each test cartridge into the containment bottle;

14) Pick up a 110 μl of Tris from cavity 55 and dispense into test cartridge; repeat for each test cartridge;

15) Aspirate Tris from each test cartridge into the containment bottle;

16) Pick up 150 μl distilled H₂O from cavity 59 and dispense into test cartridge; repeat for each test cartridge;

17) Aspirate distilled H₂O from each test cartridge into the containment bottle;

18) Pick up 150 μl distilled H₂O from cavity 67 and dispense into test cartridge; repeat for each test cartridge;

19) Aspirate distilled H₂O from each test cartridge into the containment bottle;

20) Pick up 150 μl distilled H₂O from cavity 67 and dispense into test cartridge; repeat for each test cartridge;

21) Aspirate distilled H₂O from each test cartridge into the containment bottle;

22) Pick up 170 μl Hydroquinone from cavity 57 and dispense into test cartridge; repeat for each test cartridge;

23) Pick up 45 μl Silver Lactate from cavity 69 and dispense into test cartridge; repeat for each test cartridge;

24) Mix solution in each test;

25) Incubate each test cartridge 10 minutes;

26) Pick up 170 μl distilled H₂O from cavity 71;

27) Aspirate needles into containment bottle;

28) Pick up 170 μl distilled H₂O from cavity 71 and dispense into test cartridge; repeat for each test cartridge;

29) Aspirate distilled H₂O from each test cartridge into the containment bottle;

30) Pick up 200 μl distilled H₂O from cavity 73 and dispense into test cartridge; repeat for each test cartridge;

31) Aspirate distilled H₂O from each test cartridge into the containment bottle;

32) Dry test cartridges for 10 minutes;

Following the transfer of reagents and incubation periods required by the testing procedure, the carousel 12 indexes the test cartridges 18 for engagement with the spring loaded electrical contacts 26. The contacts 26 engage the electrical contacts 105 on each resistor sheet 103 in the test cartridge 18 to obtain a final reading prior to testing. In the preferred embodiment, an electrical current is applied to each of the three circuits containing a single resistor to determine whether the circuit is open or closed for each resistor sheet. If the circuit is closed, the final resistance in the circuit is measured.

If the test is positive, that is the final resistance has dropped a defined percentage from the base reading, a binding reaction occurred which is described fully in U.S. Pat. No. 5,284,748 and fully incorporated herein by reference. In this example, the binding reaction as evidenced by the closed circuit provides an indication of the presence of Escherichia coli in the test specimen.

While there has been shown and described what are at present considered the preferred embodiment of the invention, it will be apparent to those skilled in the art that various changes and modifications can be made therein without departing from the scope of the invention defined by the appended claims.

We claim:

1. A testing kit for use in an immunoassay testing device that detects a binding reaction between a pair of first and second substances, particularly biogenic substances, which specifically bind together allowing deposition of electrically conductive particles which fully or partially complete an essentially open electrical circuit, said device having a conveying mechanism with a plurality of predetermined container positions, said kit comprising:
a first container receivable by said conveying mechanism having a plurality of cavities for receiving fluids, at least one of said fluids is one of said substances;
at least one second container receivable by said conveying mechanism to be displaced along said axis of motion of said conveying mechanism, said second container having a cavity and having electrical contacts for engagement from above when fit in said conveying mechanism, said electrical contacts being open ends of said circuit;
whereby, said testing device transfers at least one of said fluids from said first container to said second container causing a binding reaction in the presence of said other substance.

2. A testing kit as in claim 1 wherein said containers fit together to form fit within a segment of an annulus.

3. A testing kit as in claim 1, wherein said container is adjacent to said first container when it is in said conveying mechanism.

4. A first container as in claim 1 further comprising a film covering and sealing all of said cavities in said top.

5. A first container as in claim 4, wherein said film is sized and of a material to be penetratable by a needle.

6. A first container as in claim 4, wherein said film is metal foil.

7. A first container as in claim 1 wherein at least one of said cavities contains a diluting fluid for cleansing a needle of said testing device.

8. A first container as in claim 7, wherein said plurality of cavities is 18 cavities, wherein 12 cavities are arranged in a first row of six cavities and a second row of six cavities, said remaining cavities being arranged in a third row interposed between said first row and said second row of said cavities.

9. A first container as in claim 7, wherein said cavities are nonreactive with said fluids.

10. A first container as in claim 7, wherein said cavities are polypropylene.

11. A first container as in claim 7, wherein said cavities are polyethylene.

12. A first container as in claim 7, wherein said fluids include reagents selected from a group consisting of gold-conjugated antibody, hydroquinone, Tris, distilled H₂O, and silver lactate.

13. A first container as in claim 7, wherein said base and top is non-light transmissive.

14. A first container as in claim 7, wherein said plurality of cavities have volumes between 385 microliters and 1600 microliters.

15. A first container as in claim 1, wherein said first container has a top and rear wall, said top extends past said rear wall and has at least one indent for detection by a position sensing mechanism in said testing device.

16. A second container as in claim 1 wherein said cavity has a front wall, a rear wall, a pair of opposing sidewalls, and a bottom; said electrical contacts are on a first resistor sheet having a resistor, said resistor sheet being mounted to one of said sidewalls in said cavity.

17. A second container as in claim 16, wherein said front wall and said rear wall extend inwardly and downwardly toward a receptacle at said bottom.

18. A second container as in claim 16, wherein said resistor has a resistance of at least 150 ohms.

19. A second container as in claim 16, further comprising a second resistor sheet mounted to said sidewall opposite said first resistor sheet.

20. A second container as in claim 16, wherein said resistor sheet has at least one resistor in close proximity to said bottom of said cavity, said resistor being electrically connected to said electrical contacts forming a circuit and having a layer of said other substances overlaying said resistor, said electrical contacts being at said top of said cavity, wherein said binding of said substances allows deposition of electrically conductive particles completing said circuit.

21. A second container as in claim 16, wherein said second container is formed from two halves generally symmetrical about a central plane.

22. A second container as in claim 16, wherein said top extends past said rear wall and has an indent for detection by a position sensing mechanism in said testing device.

23. A test cartridge for use in an immunoassay testing device that detects a binding reaction between a pair of first and second substances, particularly biogenic substances, which specifically bind together allowing deposition of electrically conductive particles which fully or partially complete an essentially open electrical circuit, said device having a conveying mechanism with a plurality of predetermined container positions, said test cartridge comprising:
a base having a top;
an upwardly facing cavity in said top; and
at least one essentially open electrical circuit mounted in said cavity having electrical contacts for engagement with said testing device, said electrical contacts being open ends of said circuit, whereby said electrical circuit closes upon deposition of electrically conductive particles following said binding reaction.

24. A test cartridge as in claim 23, wherein said electrical circuit is essentially open by a resistor.

25. A reagent package for use in an immunoassay testing device that detects a binding reaction between a pair of first and second substances, particularly biogenic substances, which specifically bind together allowing deposition of electrically conductive particles which fully or partially complete an essentially open electrical circuit, said device having a conveying mechanism with a plurality of predetermined container positions, said reagent package comprising:
a base having a top;
a plurality of upwardly facing cavities in said top, said cavities for receiving fluids, some of said cavities being of different volumetric capacities, at least one of said fluids is one of said substances, said cavities being nonreactive with said fluids; and
a penetrable film covering said cavities.

26. A reagent package for use in an immunoassay testing device as claimed in claim 25, wherein said fluids include reagents selected from a group consisting of gold-conjugated antibody, hydroquinone, Tris, distilled H₂O, and silver lactate.
